(19) United States
(12) Patent Application Publication
(10) Pub. No.: US 2007/0188709 A1
(43) Pub. Date: Aug. 16, 2007

(54) CORNEAL TOPOGRAPHER

(75) Inventors: Paul van Saarloos, Nedlands (AU); Mark Gallop, Nedlands (AU); Robert H. Ellelboom, Nedlands (AU)

Correspondence Address: KNOBBE MARTENS OLSON & BEAR LLP, 2040 MAIN STREET, FOURTEENTH FLOOR, IRVINE, CA, 92614, US

(73) Assignee: Clearmark Technologies Pty, Ltd., Nedlands (AU)

(21) Appl. No.: 11/735,956

(22) Filed: Apr. 16, 2007

Related U.S. Application Data
(63) Continuation of application No. 10/535,726, filed on Feb. 21, 2006.

(30) Foreign Application Priority Data
Nov. 20, 2002 (AU) 2002952772

(51) Int. Cl. A61B 3/10

(52) U.S. Cl. 351/212

(57) ABSTRACT

A corneal topographer, and a method for corneal topography are disclosed. In one embodiment, the topographer includes an illumination projection subsystem to project a series of preselected different stationary patterns of one or more slits of light in ordered succession onto the surface of the cornea. An image capture subsystem captures a still image of each projected pattern. An image processing subsystem converts the still images into topographical information of the cornea. The method involves projecting a series of preselected different stationary patterns of one or more slits of light in ordered succession onto the surface of the cornea, capturing a still image of each projected pattern and converting the still images into topographical information of the cornea.

### RELATED APPLICATIONS

This application is a continuation application, and claims the benefit under 35 U.S.C. §120 of application Ser. No. 10/535,726 filed on Feb. 21, 2006, which is hereby incorporated by reference herein.

### BACKGROUND OF THE INVENTION

1. Field of the Invention

Ophthalmologists and optometrists would like to have an accurate representation of the cornea of the eye, particularly its front surface and its thickness. This information is used to prescribe contact lenses and eye glasses, and to reshape the cornea by surgical procedures, all to improve eyesight. Since it is not possible to measure the cornea with physical objects, remote sensing techniques are used to produce this data. Corneal Topography is the name given to this field of technology, and instruments that measure corneal topography are known as corneal topography machines, or corneal topographers. This invention concerns a corneal topographer, and a method for corneal topography.

2. Description of the Related Technology

The basic and most common corneal topography systems use a Placido disk to project a series of concentric rings onto the corneal surface. The disturbance to the concentric projections is imaged by a video camera, and then complex algorithms calculate the topography. Unfortunately, this system relies on a number of assumptions and these make most measurements of irregularly shaped eyes very inaccurate. In general people who have irregularly shaped eyes also have the greatest need for accurate surgery.

A much more complex and expensive system scans narrow bands, or slits, of light across the surface of the cornea. The slits are imaged as they scan across the eye, and again complex algorithms calculate the topography. This method does not reply on any assumptions, generating accurate maps of both normal and irregularly shaped corneas. It has the added advantage of simultaneously providing data to measure the thickness of the cornea. This is because the projection of the slit produces a number of reflections as it passes through the cornea and anterior chamber of the eye.

The most common application of corneal topography is for planning refractive surgery. This surgery has been successful in correcting the vision of many millions of people worldwide. However, in almost all cases, these patients have had a simple variation or deviation from the normal corneal shape. There is a larger group of people with irregular variation in corneal shape (irregular astigmatism). These require custom control of the laser that is reshaping the cornea, which ablates varying amounts of tissue over the cornea. It of course relies on accurate topography data of this irregular astigmatism. Ideally the topography data is fed directly into the control system for the laser; this is called custom ablation.

### SUMMARY OF CERTAIN INVENTIVE ASPECTS

One aspect of the invention provides a corneal topographer, comprising: i) an illumination projection subsystem to project a series of preselected different stationary patterns of one or more slits of light onto in ordered succession the surface of the cornea, ii) an image capture subsystem to capture a still image of each projected pattern and iii) an image processing subsystem to convert the still images into topographical information of the cornea.

Such a topographer represents a relatively inexpensive device which provides accurate topographical information of the cornea without making assumptions. In particular this is achieved by the use of multiple stationary slits, and elimination of scanning, and therefore moving parts.

The light source may be collimated LEDs, masked and focused onto the eye.

In total there may be many, such as forty-eight LEDs producing the same number of slits, and these may be projected in, say, fifteen to twenty different patterns to provide sufficient data to map the topography of the cornea. There may be more or less slits; depending on the amount of resolution achieved. The size of the slits may also vary, and the draft angle.

The LEDs may be housed together in sets with a common focusing arrangement.

A CCD video camera may be used, under the control of a computer to receive the images. The computer may also control a frame grabber to capture a still image every time a new combination of slits is projected onto the cornea.

Analysis may involve registration of the whole image sequence to compensate for saccadic or other eye movements that occur in the time interval between capture of successive images.

Next, image processing may determine the two edges of the slits as they are shown on the image. The edges of the slits define the anterior and posterior surfaces of the cornea. Other reflections may be off the iris and the two surfaces of the lens of the eye.

The edges may then be converted into mathematical curves.

The curves may then be used to determine the external shape of the cornea, the inside surface of the cornea, and all the local shape variations in these surfaces. The thickness of the cornea can also be calculated. The reflections off other surfaces maybe used to calculate the volume of the anterior chamber and distances to the lens.

The topography data may be displayed.

Another aspect of the invention provides a method for corneal topography. The method involves: projecting a series of preselected different stationary patterns of one or more slits of light in ordered succession onto the surface of the cornea, capturing a still image of each projected pattern and converting the still images into topographical information of the cornea.

### BRIEF DESCRIPTION OF THE DRAWINGS

Embodiments of the invention will now be described with reference to the accompanying drawings.

FIG. 1 is a pictorial diagram of a corneal topographer having eight light emitters and a controlling computer.

FIG. 2 is a sectional view of one of the light emitters of FIG. 1; also showing the relationship between that light emitter, the camera and an eye during use of the topographer.

FIG. 3 is a diagram of an eye showing the arrangement of light slits projected onto it from the topographer of FIG. 1. provides

### DETAILED DESCRIPTION OF CERTAIN INVENTIVE EMBODIMENTS

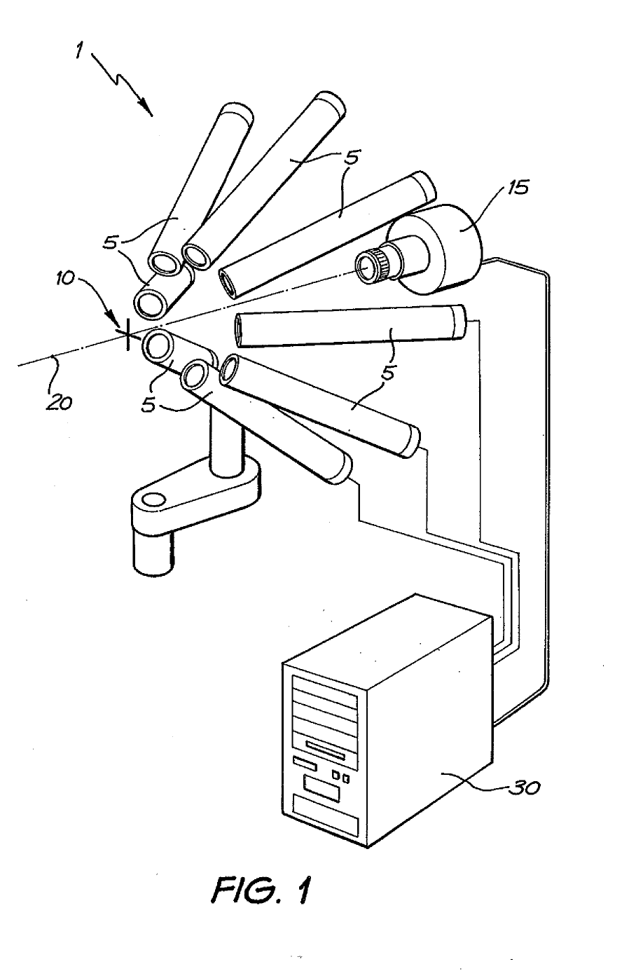

Referring first to FIG. 1, the corneal topographer 1 comprises eight light emitters 5 mounted to focus slits of light at the same point 10 from different angles. A video camera 15 is arranged with its axis 20 through point 10 to image the slits. A computer 30 controls the emitters 5 and the video camera 15.

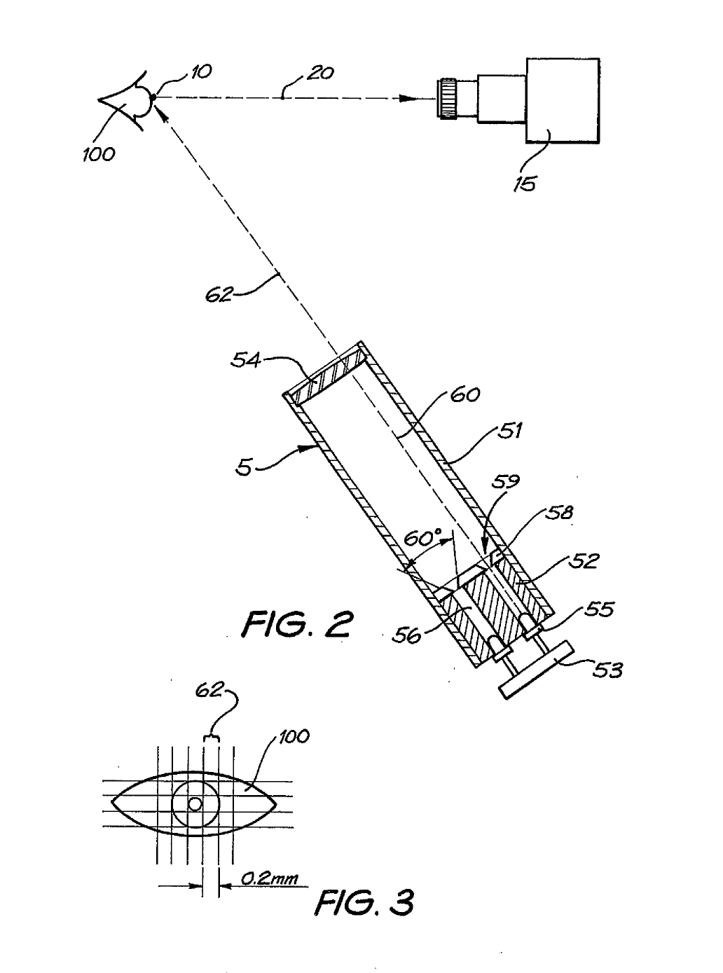

Referring now to FIG. 2, each emitter 5 comprises a tube 51 having a LED holder 52 and an electrical panel 53 at the remote end, and a lens 54 at the end closest to point 10. Six 5mm LEDs 55 are mounted in separate channels 56 through LED holder 52. A layer of stereo lithography material 58, tinted to be opaque, is mounted to the end of the LED holder 52. A distance, say 100 mm, of twice the focal length of lens 54 extends between the layer of stereo lithography material 58 and lens 54.

Each channel 56 in the LED holder 52 collimates the light from the LED mounted in that channel. Slit-like openings in the stereo lithography material at the end of each channel have a draft of 60 o to produce a knife edge slit 59 that is 0.2 mm wide. Light from LED 55 passes through knife edge slit 59 to produce a light slit 60 that is 24 mm high. Lens 54 reduces the slit of light 60 from 24 mm high to 15 mm high for projection 62 onto the eye 100; as shown in FIG. 3. Light slit 62 is a vertical slit, relative to eye 100. The image of slit 62 projected on the eye is captured by video camera 15.

In total there are forty-eight LEDs 55 and they are all wired to electrical panels 53 at the remote end of the light tubes 51 so that they can be individually turned on and off under the control of the computer indicated schematically at 30. Should all the LED's be turned on at once they would project over the entire visible surface of the cornea in a pattern of both horizontal and vertical contiguous slits.

A significant amount of time may be devoted to the positioning of the light emitters, so that the entire corneal surface will be covered. Optimal resolution will be achieved when each part of the cornea is measured by at least one slit projection.

The topographer 1 is mounted on a conventional ophthalmic assessment stand (not shown), which supports the patient's head on a head and chin rest. The device is mounted so that its position is under the control of a mechanical joystick, allowing back and forth movement for focus, left and right movement to move from one eye to the other, and smaller horizontal and vertical movements to centre the device in front of the eye being assessed.

In use, the topographer is aligned in front of the eye so that the light slits are properly focused on the cornea, and so that the camera image is in focus. A single or series of LEDs or other lights will be mounted near the camera to which the patient must fixate.

Preselected different patterns of LEDs are then sequentially turned on and off under the control of the computer. The camera 15, a CCD video camera, operates under the control of the computer to receive the light reflected along axis 20 for each pattern. The computer also controls a frame grabber to capture a still image every time a new combination of slits is projected onto the cornea. Each still image is stored as quickly as it is produced, on an image capture card.

The topographer typically produces a series of between 15 and 20 images of each eye, in a total time period less than 2 seconds. The information on the captured images is then converted into topographical information of the cornea.

This involves registration of the whole image sequence to compensate for the (very small) saccadic eye movements or eye drifts that occur in the time interval between capture of successive images. This is done by the use of one or two slits that will always be on, for instance the two slits furthest apart, and using landmarks on the eye to determine if and how far the eye has moved.

Next, image processing determines the two edges of the slits as they are shown on the image. One of the edges determines the outside surface of the cornea, the other determines the inside surface of the cornea.

The edges are converted into mathematical curves.

Using trigonometry, the data of all the curves are assembled to determine the shape of the cornea, the inside surface of the cornea, and all the local shape variations in these surfaces. The thickness of the cornea can also be calculated.

The software then displays the topography data in various forms. For instance, the corneal data will be displayed as a series of color coded providing axial, refractive, elevation and irregularity data. Other functions include showing a live view of the cornea before imaging to position the device, and export of data for control of refractive lasers during surgery. Standard data handling functions are also performed, such as storage with patients' data, archiving, comparison between sessions, and printing.

Although the invention has been described with reference to a particular example, it should be appreciated that it includes many variants and alternatives. For instance, 3 mm LEDs could be used, and this could lead to the use of fewer light emitters. Fewer or more LEDs may be used with differing resolution requirements. The slits may be projected at varying angles; not only horizontal and vertical.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

What is claimed:

1. A corneal topographer, comprising:
an illumination projection subsystem configured to project a series of preselected different stationary patterns of one or more slits of light in ordered succession onto the surface of the cornea;
an image capture subsystem configured to capture an image of each projected pattern; and
an image processing subsystem to convert the images into topographical information of the cornea.

2. A corneal topographer according to claim 1, wherein the illumination projection subsystem makes use of collimated LEDs, masked and focussed onto the eye.

3. A corneal topographer according to claim 2, wherein there are up to fort-eight LEDs producing the same number of slits.

4. A corneal topographer according to claim 1, wherein the slits are projected in up to twenty different patterns.

5. A corneal topographer according to claim 2, wherein the LEDs are housed together in sets with a common focussing arrangement.

6. A corneal topographer according to anyone of claims 1, wherein a CCD, video camera is used under the control of a computer to capture the images.

7. A corneal topographer according to claim 6, wherein the computer also controls a frame grabber to capture a still image every time a new combination of slits is projected onto the cornea.

8. A corneal topographer having illumination, image capture and image processing subsystems, wherein analysis involves registration of the whole image sequence to compensate for saccadic or other eye movements that occur in the time interval between capture of successive images;
next, image processing determines the two edges of the slits as they are shown on the image;
the edges are then converted into mathematical curves; and
the curves are then used to determine the external shape of the cornea, the inside surface of the cornea, and all the local shape variations in these surfaces.

9. A corneal topographer according to claim 8, wherein the thickness of the cornea is also calculated.

10. A corneal topographer according to claim 8, wherein reflections off other surfaces are used to calculate the volume of the anterior chamber and distances to the lens.

11. A corneal topographer according to claim 1, farther comprising: a display configured to display the topography data.

12. A method of corneal topography, comprising:
projecting a series of preselected different stationary patterns of one or more slits of light in ordered succession onto the surface of the cornea;
capturing an image of each projected pattern; and
converting the images into topographical information of the cornea.

13. A method according to claim 12, wherein analysis involves registration of the whole image sequence to compensate for saccadic or other eye movements that occur in the time interval between capture of successive images.

14. A method according to claim 12, wherein image processing determines the two edges of the slits as they are shown on the image.

15. A method according to claim 14, wherein the edges are converted into mathematical curves.

16. A method according to claim 15, wherein the curves are used to determine the external shape of the-cornea, the inside surface of the cornea, and all the local shape variations in these surfaces.

17. A method according to claim 16, wherein the thickness of the cornea is also calculated.

18. A method according to claim 16, wherein reflections off other surfaces are used to calculate the volume of the anterior chamber and distances to the lens.

19. A method according to claim 12, further displaying the topography data.

20. A corneal topographer, comprising:
means for projecting a series of preselected different stationary patterns of one or more slits of light in ordered succession onto the surface of the cornea;
means for capturing an image of each projected pattern; and
means for converting the images into topographical information of the cornea.
